(19) United States
(12) Patent Application Publication
(10) Pub. No.: US 2022/0265032 A1
(43) Pub. Date: Aug. 25, 2022

(54) Universal Wearable Sanitizer Receptacle

(71) Applicant: Dorothy E. Datcher, Pell City, AL (US)

(72) Inventor: Dorothy E. Datcher, Pell City, AL (US)

(21) Appl. No.: 17/100,497

(22) Filed: Nov. 20, 2020

(51) Int. Cl. A45F 5/02; A61L 2/26; A61L 2/00

(52) U.S. Cl. CPC A45F 5/021; A61L 2/26; A61L 2/0088; A61L 2202/16

(57) ABSTRACT

Disclosed is a universal wearable sanitizer receptacle that comprises a rear panel and pouch joined to said rear panel. The rear panel allows for use of a belt loop, a clip and or an adjustable strap that a user can wear around their neck, shoulder or waist. The rear panel top edge has a slot that allows for installation of a removable clip. Located on the front of the pouch is a first pocket and located on the front of the first pocket is a second pocket with a transparent front panel.

### FIELD OF THE INVENTION

The present invention relates to a wearable and portable sanitizer carrying receptacle that may be worn in a number of different ways dependent upon how the user so wishes to carry the sanitizer receptacle.

### BACKGROUND OF THE INVENTION

Currently, the world has been disrupted by the novel corona virus COVID-19. There are a number of precautions individuals can take to prevent contact with the virus. One of these precautions is providing for clean and disinfected hands after touching potentially unclean surfaces. To achieve this, a person must use an appropriate sanitizer that has been proven to kill the unwelcome virus. Most of these sanitizers are in liquid form and require a dispenser to adequately meter out an appropriate amount of sanitizer onto a person's hands. Oftentimes, as individuals go about their daily lives running errands, working and taking part in other activities, it can be cumbersome to carry around hand sanitizer. Women generally carry purses, but it is still cumbersome to find hand sanitizer in a purse or, as is often the case, many women's purses are not big enough to hold the dispenser or that their purses already contain many items such there is not adequate room. Most men do not carry purses and pants pockets are generally not adequate to house most hand sanitizer dispensers. Even if the sanitizer dispenser is small in nature, most men would prefer not to carry around sanitizer in their pockets in addition to keys, wallets and other items. What is needed in the art is a universal wearable sanitizer receptacle that can house a sanitizer dispenser and other small valuables and can be worn in a variety of fashions my both men and women. Further, what is needed in the art is sanitizer receptacle that house a sanitizer bottle and allow for dispensing of sanitizer without having to remove the sanitizer bottle from the sanitizer receptacle. A further need in the art is a sanitizer receptacle that allows one hand operation without having to come into contact with a sanitizer bottle. Another need in the art is a sanitizer receptacle that a user can wear but also allows other individuals around them to access the sanitizer bottle and dispense sanitizer without the wearer of the receptacle having to transfer by hand the sanitizer bottle other individuals around them.

### SUMMARY OF THE INVENTION

The present invention is a universal wearable sanitizer receptacle comprising a rear panel that has attached onto its front surface a pouch adapted to receive at least one of a sanitizer bottle and/or a hand washing detergent. The rear panel further has an aperture in the upper right corner and an aperture in the upper left corner adapted to receive a strap worn by a user. Also disclosed is a first pocket located on the front of the pouch to accept small items such as a credit card, driver's license and other small items like currency. Directly on front of the first pocket is a second pocket with a clear or transparent plastic panel that allows a user to identify their receptacle with an insert that displays the their name, initials and/or a logo or other such indicia of their choice. Further disclosed on the back of the rear panel is a belt loop adapted to be inserted onto a user's belt or purse belt. Additionally, there is a belt clip that is attachable to the rear panel and removable when a person prefers to use one of the various other ways to wear the receptacle.

### BRIEF DESCRIPTION OF THE DRAWINGS

FIG. 1 is a front perspective view of the universal wearable sanitizer receptacle.

FIG. 2 is a rear perspective view elevation of the universal wearable sanitizer receptacle.

FIG. 3 is a side elevation of alternate embodiment of the sanitizer receptacle.

FIG. 4 is a cross sectional view of the universal wearable sanitizer receptacle along the line 4-4 as shown in FIG. 2.

FIG. 5 is a front elevation view of the universal wearable sanitizer receptacle.

FIG. 6 is a rear elevation view of the universal wearable sanitizer receptacle.

### DETAILED DESCRIPTION

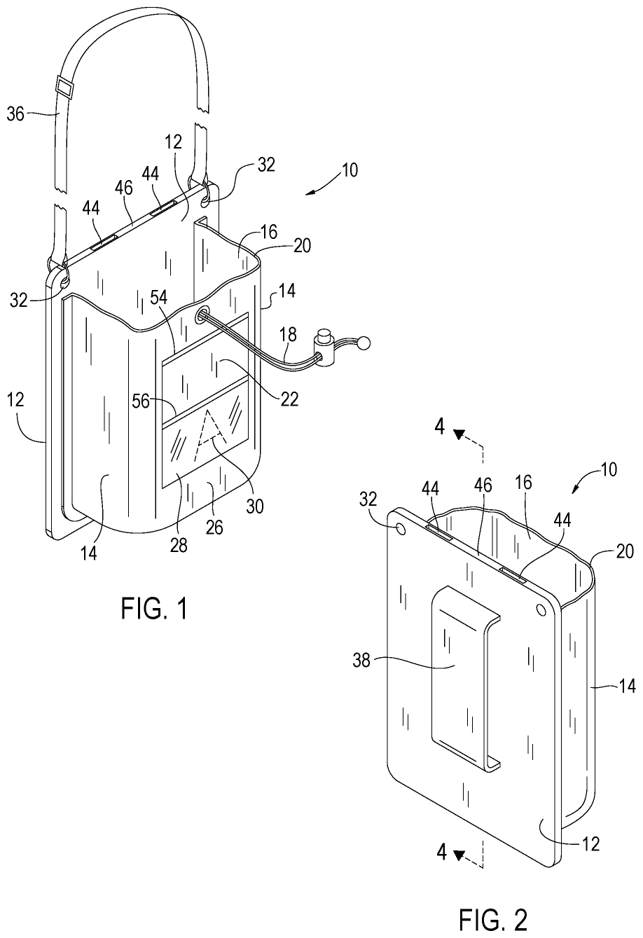

Referring now to FIGS. 1-2 there is shown a universal wearable sanitizer receptacle 10 hereinafter interchangeably referred to as the “receptacle”. There is shown a rear panel 12 having a pouch 14 on said rear panel's 12 front face whereby said pouch 14 is either glued, sewn, or thermally fused onto said rear panel 12 front face. Said pouch 14 has an upper aperture 16 that is adapted to receive a sanitizer bottle 11. The upper aperture 16 allows for a wide variety of sanitizer bottles 11 and especially is directed to using a pump style sanitizer bottle 11. The pouch 14 upper aperture 16 has an upper edge 20 and contained within said upper edge 20 is a drawstring 18. The drawstring 18 and upper edge 20 combination allows for the pump apparatus of a sanitizer bottle 11 to remain exposed and ready for use at will but allows for securing of the sanitizer bottle 11 by means of the drawstring 18 and upper edge 20 of the pouch 14 constricting around the neck of the sanitizer bottle 11 thereby maintaining tight connection. This allows a user to dispense sanitizing fluid without having to remove the sanitizer bottle. The rear panel 12 also has apertures 32 located on the left and right upper corners of the rear panel 12. The apertures 32 are purposed for use with an adjustable strap 36 such that each end of the adjustable strap 26 attaches to said apertures 32 such that the receptacle 10 can be worn around a user's neck, over a user's shoulder or even attached to a user's waist whereby the adjustable strap 26 can function like a belt in a situation whereby a user's clothing choice does not provide for wearing of a belt. As shown in FIG. 2, there is located a belt loop 38 on the back of the rear panel 12 rear face. This allows a user who is wearing a belt to secure the receptacle 10 onto their waist via said belt loop 38.

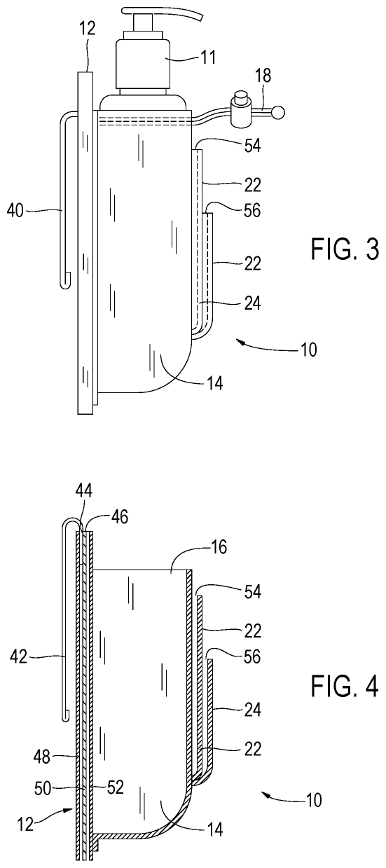

Further shown in FIGS. 1-2 and 4 is a slot 44 located in the top edge 46 of the rear panel 12 which is purposed for accepting a removable clip 42 as shown in FIGS. 4 and 6. The removable clip 42 allows for a user to clip the receptacle 10 onto a waist band of a user when that user is not wearing a belt or said clothing choice does not provide use with a belt. The removable clip 42 also allows the receptacle 10 to be attached to other items such as the side of a purse, a pants pocket and other thin edges where it could find purchase. The removability of the clip 42 is desirable when a user chooses to use the belt loop 38 as opposed to the removable clip 42 and the removal of said clip 42 prevents the clip 42 from digging in and/or chaffing the waist of a user. When not in use, the removable clip 42 can be stored in the bottom of the pouch 16. Referring now to FIG. 3 there is shown an alternate embodiment of the receptacle 10 whereby there is a clip 40 joined to the rear face of the rear panel 12 instead of a belt loop.

Referring now to FIG. 4 there is shown a cross-sectional view of the receptacle 10 revealing that the rear panel is comprised of a rear fabric sheet 48, a semirigid planar sheet 50 and a front fabric sheet 52. For purposes of this invention and the present disclosure use of the word “fabric” can mean cloth made from any number of materials from which clothing is made including cotton, polyester, nylon, rayon, etc., and also neoprene, plastic, rubber, leather, synthetic leather, vinyl and other similar planar flexible materials. In a preferred embodiment, the fabric used to form the rear panel 12 and pouch 14 would have anti-microbial properties. The semirigid planar sheet 50 in a preferred embodiment would be made of a plastic material that provides rigidity to the rear panel but also is flexible to conform to the waist of a user when they perform various movements such as bending over, to the side and/or contorting their body for whatever the purpose. The semirigid planar sheet 50 also provides the rear panel 12 structural support when the removable clip 42 is inserted into the slot 44 located on the top edge of the rear panel 12. The semirigid planar sheet 50 is located in the middle of the rear fabric sheet 48 and the front fabric sheet 52. The rear fabric sheet 48 and the front fabric sheet 52 can either be separate pieces of fabric joined to each other at the their bottom and top edge and left and right edges or they can be one long rectangular piece of fabric that is folded onto itself such that each of the rectangular fabric ends touches each other and forms the top edge and whereby the left and right edges are joined and/or sewn together. The top edge 46 in one embodiment is partially joined whereby the slot 44 is formed to accommodate the removable clip 42. Additionally, an additional slot 44 can be located on the top edge 46 opposite the other slot such that the receptacle 10 is versatile for left handed and right handed individuals or to accommodate particular situations based on what a user is also wearing on their belt or waistband. Further, an additional removable clip 42 can be employed such that two clips can be used to engage with a user's belt or waist band to provide additional securement.

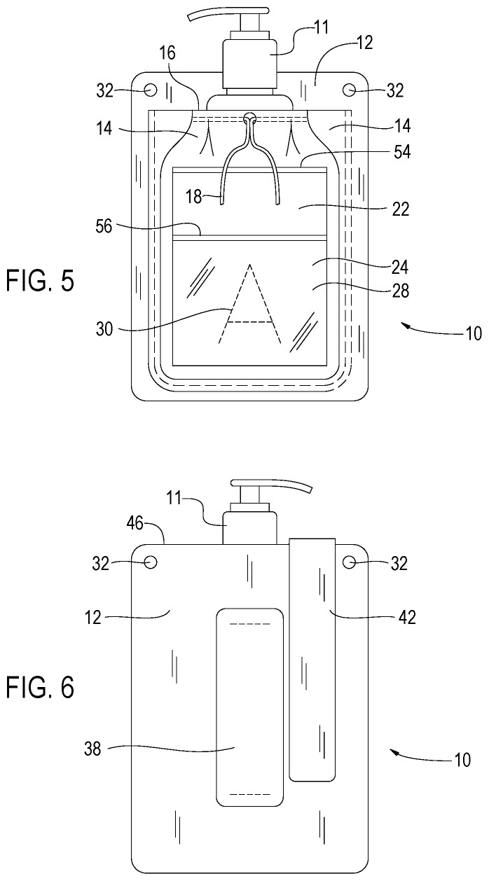

Referring now to FIGS. 1, 3-4 and 5 there is shown a first pocket 22 that is joined onto the front face of the pouch 14 whereby the first pocket 22 is directly adjacent and parallel with the front of the pouch 14. The first pocket 22 depth is much smaller than the height or the width of the first pocket 22 is designed to hold thin items such as credit cards, identification cards, membership cards and/or cash. The first pocket 22 is made of fabric. The first pocket 22 is not readily observable to an observer at a distance, but it is only apparent upon close inspection. At the top of the first pocket 22 is a slot 54 that allows the aforesaid items to be inserted. Located on the front of the first pocket 22 is located a second pocket 24 that is joined onto the front face of the first pocket 22. The second pocket 24 is directly adjacent and parallel with the front face of the first pocket 22. The second pocket's 24 front face is a transparent panel 28 that is designed to hold a decal or other planar item showing an indicia 30 such as a logo, writing, picture, symbol or other printed or photographed material. The transparent panel 28 allows for interchangeable indicia 30 to be introduced into a slot 56 of the second pocket 24 and to be viewable to observers. The transparent panel 28 is made of a flexible clear material such as plastic, vinyl, polymeric cellulose film, polyethylene and other flexible clear materials.

The principles, embodiments, and modes of operation of the present invention have been set forth in the foregoing specification. The embodiments disclosed herein should be interpreted as illustrating the present invention and not as restricting it. The foregoing disclosure is not intended to limit the range of equivalent structure available to a person of ordinary skill in the art in any way, but rather to expand the range of equivalent structures in ways not previously contemplated. Numerous variations and changes can be made to the foregoing illustrative embodiments without departing from the scope and spirit of the present invention.

### ENUMERATED ELEMENTS

10 Universal wearable sanitizer receptacle
11 Sanitizer bottle
12 Rear panel
14 Pouch
16 Upper aperture
18 Drawstring
20 upper edge
22 First pocket
24 Second pocket
26 Front face
28 Transparent panel
30 Indicia
32 Apertures
36 Adjustable strap
38 Belt loop
40 Clip
42 Removable clip
44 Slot
46 Top edge
48 Rear fabric sheet
50 Semirigid planar sheet
52 Front fabric sheet
54 first pocket slot
56 second pocket slot

What I claim is:

1. A universal wearable sanitizer receptacle, comprising:
a rear panel having a left aperture in the upper left corner of said rear panel and a right aperture in the upper right corner of said rear panel;
a pouch located on a front face of the rear panel and said pouch adapted to receive a sanitizer bottle into an upper aperture;
a belt loop or a clip located on a rear face of the rear panel;
a first pocket located on the front surface of the pouch having a slot for insertion of various articles;
a second pocket located on the front surface of said first pocket and said second pocket further comprising a transparent panel as its front surface and said second pocket having a slot for insertion of an indicia bearing medium that bears an indicia; and
an adjustable strap adapted to connect to said left aperture and right aperture of the rear panel.

2. The universal wearable sanitizer receptacle of claim 1 wherein the rear panel is comprised of rear fabric sheet, a semi-rigid planar sheet and a front fabric sheet and whereby the rear fabric sheet is joined to the front fabric sheet and the semi-rigid planar sheet is encapsulated between the rear fabric sheet and the front fabric sheet.

3. The universal wearable sanitizer receptacle of claim 2 further comprising a slot formed in the top edge of the rear panel and adapted to receive a removable clip.

4. The universal wearable sanitizer receptacle of claim 3 further comprising a second slot formed in the top edge of the rear panel opposite the slot and adapted to receive a second removable clip.

5. The universal wearable sanitizer receptacle of claim 4 wherein the pouch and rear panel are made of an anti-microbial fabric.

6. The universal wearable sanitizer receptacle of claim 5 wherein there is a drawstring located in the vicinity of an upper edge of the pouch and whereby the drawstring will cinch the upper edge of the pouch around a sanitizer bottle and secure the sanitizer bottle in place.

7. The universal wearable sanitizer receptacle of claim 1 further comprising a slot formed in the top edge of the rear panel and adapted to receive a removable clip.

8. The universal wearable sanitizer receptacle of claim 7 further comprising a second slot formed in the top edge of the rear panel opposite the slot and adapted to receive a second removable clip.

9. The universal wearable sanitizer receptacle of claim 1 wherein the pouch and rear panel are made of an anti-microbial fabric.

10. The universal wearable sanitizer receptacle of claim 1 wherein there is a drawstring located in the vicinity of an upper edge of the pouch and whereby the drawstring will cinch the upper edge of the pouch around a sanitizer bottle and secure a sanitizer bottle in place.

11. A universal wearable sanitizer receptacle, comprising:
a rear panel having a left aperture in the upper left corner of said rear panel and a right aperture in the upper right corner of said rear panel;
a pouch located on a front face of the rear panel and said pouch adapted to receive a sanitizer bottle into an upper aperture;
a belt loop or a clip located on a rear face of the rear panel;
a first pocket located on the front surface of the pouch having a slot for insertion of various articles;
an indicia printed or stitched onto the front of the first pocket; and
an adjustable strap adapted to connect to said left aperture and right aperture of the rear panel.

12. The universal wearable sanitizer receptacle of claim 11 wherein the rear panel is comprised of rear fabric sheet, a semi-rigid planar sheet and a front fabric sheet and whereby the rear fabric sheet is joined to the front fabric sheet and the semi-rigid planar sheet is encapsulated between the rear fabric sheet and the front fabric sheet.

13. The universal wearable sanitizer receptacle of claim 12 further comprising a slot formed in the top edge of the rear panel and adapted to receive a removable clip.

14. The universal wearable sanitizer receptacle of claim 13 further comprising a second slot formed in the top edge of the rear panel opposite the slot and adapted to receive a second removable clip.

15. The universal wearable sanitizer receptacle of claim 14 wherein the pouch and rear panel are made of an anti-microbial fabric.

16. The universal wearable sanitizer receptacle of claim 15 wherein there is a drawstring located in the vicinity of an upper edge of the pouch and whereby the drawstring will cinch the upper edge of the pouch around a sanitizer bottle and secure the sanitizer bottle in place.

17. The universal wearable sanitizer receptacle of claim 11 further comprising a slot formed in the top edge of the rear panel and adapted to receive a removable clip.

18. The universal wearable sanitizer receptacle of claim 17 further comprising a second slot formed in the top edge of the rear panel opposite the slot and adapted to receive a second removable clip.

19. The universal wearable sanitizer receptacle of claim 11 wherein the pouch and rear panel are made of an anti-microbial fabric.

20. The universal wearable sanitizer receptacle of claim 1 wherein there is a drawstring located in the vicinity of an upper edge of the pouch and whereby the drawstring will cinch the upper edge of the pouch around a sanitizer bottle and secure a sanitizer bottle in place.
